SEQUENCE LISTING (3 sequences)

SEQ ID NO: 1 - DNA, 1419 bp, synthetic construct
  misc_feature 1..1419: note = The sequence is synthetized.
  source 1..1419: mol_type = other DNA
gccttaacatgcaagtcgaacggcagcacgggtgcttgcacctggtggcgagtggcgaacgggtgagtaatacatcggaacatgtcctgtagtgggggatagcccggcgaaagccggattaataccgcatacgatctacggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgaaagcctgatccagcaatgccgcgtgtgtgaagaaggccttcgggttgtaaagcacttttgtccggaaagaaatccttggctctaatacagtcgggggatgacggtaccggaagaataagcaccggctaactacgtgccagcagccgcggtaatacgtagggtgcgagcgttaatcggaattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaatcctgctgagaggcgggagtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggttgccaacccgcgagggggagctaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtag

SEQ ID NO: 2 - DNA, 20 bp, synthetic construct
  misc_feature 1..20: note = The sequence is synthetized.
  source 1..20: mol_type = other DNA
agagtttgatcctggctcag

SEQ ID NO: 3 - DNA, 22 bp, synthetic construct
  misc_feature 1..22: note = The sequence is synthetized.
  source 1..22: mol_type = other DNA
tacggctaccttgttacgactt

(19) United States
(12) Patent Application Publication
(10) Pub. No.: US 2023/0088888 A1
(43) Pub. Date: Mar. 23, 2023

(54) PHOSPHORUS-DISSOLVING BACTERIUM RBC25 REGULATED BY PHOSPHATE DEFICIENCY AND APPLICATION THEREOF

(71) Applicant: Fujian Agriculture and Forestry University, Fuzhou (CN)

(72) Inventors: Yongjia ZHONG, Fuzhou (CN); Hong LIAO, Fuzhou (CN)

(73) Assignee: Fujian Agriculture and Forestry University, Fuzhou (CN)

(21) Appl. No.: 17/934,206

(22) Filed: Sep. 22, 2022

(30) Foreign Application Priority Data
Sep. 22, 2021 (CN) 202111108267.9

(51) Int. Cl. C12N 1/20

(52) U.S. Cl. CPC C12N 1/205; C12R 2001/38

(57) ABSTRACT

A phosphorus-dissolving bacterium RBC25 regulated by Phosphate deficiency and application thereof are provided. The phosphorus-dissolving bacterium RBC25 is obtained by isolating from roots of soybean planted in acidic soil. The phosphorus-dissolving bacterium RBC25 belongs to Burkholderia sp., which has a dissolving capacity for insoluble inorganic phosphate and organic phosphate. The phosphorus-dissolving bacterium RBC25 is regulated by phosphate deficiency, which can effectively colonize on soybean roots under phosphate-deficient conditions, and thus promote soybean growth under low-phosphate conditions. The phosphorus-dissolving bacterium RBC25 releases dissolved phosphate by dissolving insoluble inorganic phosphate and organic phosphate, so that it helps plants to absorb insoluble phosphate. Compared with the non-inoculated control, inoculating RBC25 in greenhouses can increase the biomass and phosphorus content of soybean by 39.6% and 35.4%, respectively, and inoculating RBC25 in fields can increase the biomass and phosphorus content of soybean by 12.5% and 16.9%, respectively.

### CROSS REFERENCE TO THE RELATED APPLICATIONS

The application is based upon and claims priority to Chinese Patent Application No: 202111108267.9, filed on Sep. 22, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML, copy is named GBYC067_Sequence_Listing.xml, created on Sep. 19, 2022, and is 6,139 bytes in size.

### TECHNICAL FIELD

The present invention belongs to the field of microbial technology, and more particularly relates to a phosphorus-dissolving bacterium RBC25 regulated by Phosphate deficiency and the application thereof.

### BACKGROUND

Phosphorus is second only to nitrogen in importance for plants, and it is one kind of the mineral nutrition essential for plants growth and development. Plants mainly absorb phosphorus from the soil in the form of phosphate through the root system. However, phosphate in soil is easily fixed by cations such as calcium ion, iron ion, aluminum ion and other cations in soil, or forms organic phosphorus, it cannot be directly absorbed and utilized by plants, resulting in extremely low concentration of phosphorus in soil that can be absorbed and utilized by plants, which is an important factor limiting plant growth. Evolutionarily, plants can promote the absorption and utilization of phosphorus in soil through the interaction with microorganisms in soil. Therefore, the separation and application of phosphate-solubilizing microorganisms in soil that can effectively colonizeplant on plant roots is an effective way to improve the absorption and utilization of phosphorus in soil and promote plants growth.

### SUMMARY

One object of the present invention is to provide a phosphorus-dissolving bacterium RBC25 regulated by Phosphate deficiency and the application thereof.

The objects of the present invention are achieved by the following technical solution:

The present invention also provides a phosphorus-dissolving bacterium RBC25 regulated by a Phosphate deficiency, wherein the phosphorus-dissolving bacterium RBC25 is isolated from roots of a soybean planted in an acidic soil and is taxonomically classified as Burkholderia sp. RBC25; wherein the phosphorus-dissolving bacterium RBC25 is deposited on Jun. 28, 2021 in the China Center for Type Culture Collection in Wuhan University, Wuhan City, Hubei Province with a deposit number of CCTCC NO: M2021796.

Cultivation and morphological characteristics of phosphorus-dissolving bacterium RBC25 as follows: The phosphorus-dissolving bacterium RBC25 can grow on TSB medium and ordinary LB medium. The phosphorus-dissolving bacterium RBC25 can form faint yellow, sheen, round and well-defined colonies on solid medium.

Physiological and biochemical characteristics of phosphorus-dissolving bacterium RBC25 as follows: The phosphorus-dissolving bacterium RBC25 can grow on solid medium containing insoluble inorganic phosphorus or insoluble organic phosphorus, and can produce a transparent phosphorus dissolving circle around colonies.

The phosphorus-dissolving bacterium RBC25 mentioned above is regulated by phosphate deficiency and is configured to be enriched in soybean roots under phosphate deficient conditions.

The present invention also provides the application of phosphorus-dissolving bacterium RBC25 in promoting soybean growth.

The present invention has the following beneficial effects:

The phosphorus-dissolving bacterium RBC25 regulated by Phosphate deficiency not only has the ability to dissolve calcium phosphorus, aluminum phosphorus and mineralized organic phosphorus phytic acid, but also can existing in the soybean roots. The abundance of RBC25 is regulated by soybean phosphorus signaling, and phosphate deficiency can induce RBC25 to colonize in soybean roots. The phosphorus-dissolving bacterium RBC25 can improve the absorption and utilization of phosphorus and promote the growth of soybean by dissolving phosphorus and mineralizing organic phosphorus. Compared with the non-inoculated control, inoculating RBC25 in greenhouses can increase the biomass and phosphorus content of soybean by 39.6% and 35.4% respectively, inoculating RBC25 in fields can increase the biomass and phosphorus content of soybean by 12.5% and 16.9% respectively.

### BRIEF DESCRIPTION OF THE DRAWINGS

FIGS. 1A-1C. Phosphorus-dissolving function identification of phosphorus-dissolving bacterium RBC25. FIG. 1A: calcium phosphorus; FIG. 1B: phytate phosphorus; FIG. 1C: aluminum phosphorus.

FIG. 2. Evolutionary tree of phosphorus-dissolving bacterium RBC25.

FIG. 3. Colonization analysis of RBC25(marked by fluorescence) in soybean roots.

FIG. 4. Low phosphorus promotes the colonization of RBC25 in soybean roots. BF: White light; GFP: Green channel; LP: Low phosphorus condition; HP: High phosphorus condition.

FIG. 5: Effect of inoculating strain RBC25 under indoor potted condition for biomass and P nutrient absorption in soybean.

FIGS. 6A-6C: Effect of inoculating strain RBC25 under field condition for biomass and P nutrient absorption in soybean. FIG. 6A: The process of soybean; FIG. 6B: Biomass; FIG. 6C: P nutrient absorption.

### DETAILED DESCRIPTION OF THE EMBODIMENTS

In order to make the contents of the present invention easier to understand, the present invention will be further described with reference to the specific examples below. It should be understood that these examples are only used to illustrate the present invention, not to limit the scope of the present invention.

### EXAMPLE 1

### Screening of Strain RBC25

### (1) Strain Acquisition

Brazilian 10 (Bx10) soybean roots planted in acid red soil in the field (experimental base of root biology research center, BOLUO County, Huizhou City, Guangdong Province) were collected. The root surface soil was washed with PBS buffer, and then the washed samples were disinfected with 7 vol % alcohol (1 min) and 2 VOL % sodium hypochlorite solution (5 min). The surface-sterilized samples were washed 5 times with sterile water, and then sterilized filter paper was used to absorb the water. 5 ml of sterile water was added to each sample and homogenized by a tissue lyser. The homogenate mother liquor was preliminarily filtered, diluted 10000 times with sterile water, and 100 μL were respectively coated on two kinds of bacterial solid culture media(Table 1). The medium plate was placed in a 28° C. incubator, and single colonies were picked up from the third day after coating, and purified by scribing. Monoclonal antibodies were selected and cultured in the corresponding liquid medium, and equal volumes were added 50 vol % sterile glycerin, mix well, and store the stored strains at −80° C.

TABLE 1
Formulation of two kinds of bacterial solid culture media

| Medium | Compound | Amount/L | |
|---|---|---|---|
| Trypic Soy Broth Medium | Casein | 17 | g |
| (TSB solid medium) | Soya peptone (papaic digest) | 3 | g |
| | NaCl | 5 | g |
| | K₂HPO₄ | 2.5 | g |
| | Dextrose | 2.5 | g |
| | (Agar) | 20 | g |
| | pH: 7.0 | | |
| M408 solid medium | Yeast extract | 1 | g |
| | Mannitol | 10 | g |
| | K₂HPO₄ | 0.5 | g |
| | MgSO_{4 •} 7H₂O | 0.2 | g |
| | NaCl | 0.1 | g |
| | (Agar) | 20 | g |
| | pH: 7.0 | | |

### (2) Strain Screening and Identification of Phosphate-Dissolving Ability

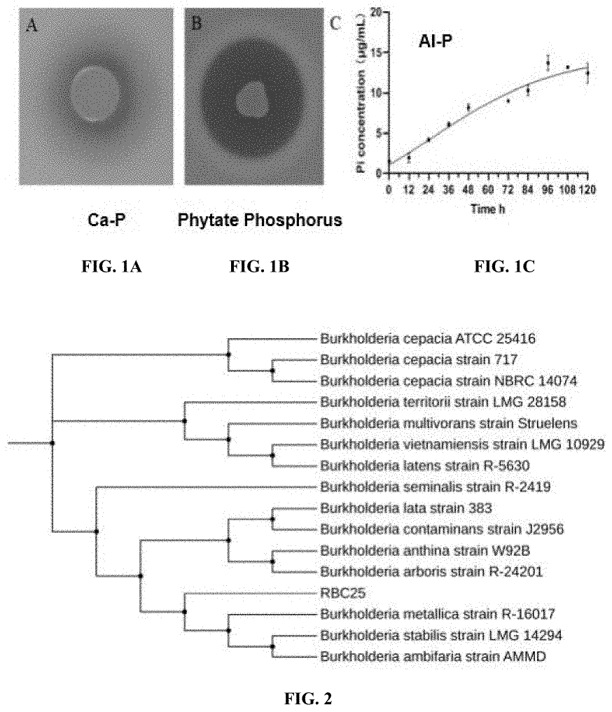

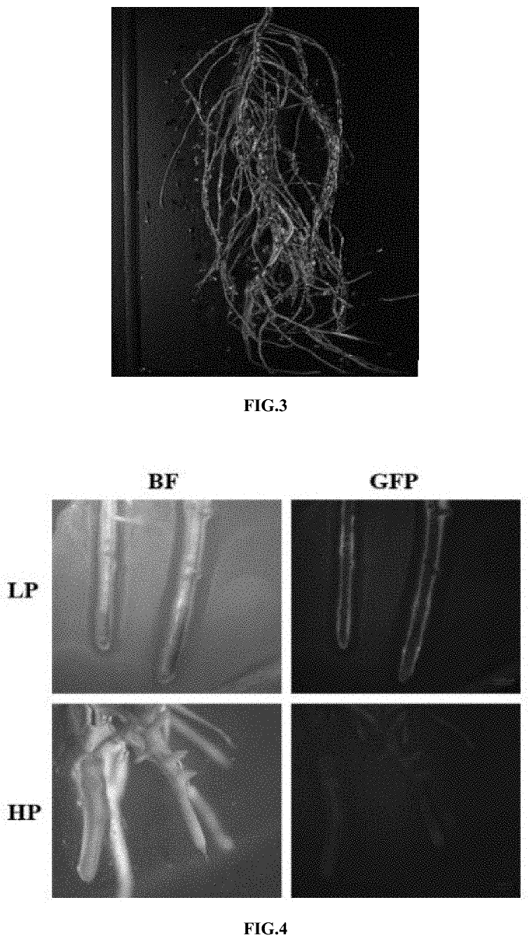

The phosphorus dissolving function of the bacteria isolated from soybean roots was tested: In an overtake clean work house, 2 μl of the preserved bacterial solution was sucked and inoculated on mengjinna calcium phytate (organic phosphorus) medium plate or mengjinna tricalcium phosphate (inorganic phosphorus) medium plate (See Table 2 for medium formulation), cultured at 30° C. for 3-5 days, growth state of strain was observed during culture. The appearance of phosphorus dissolving circle indicates that the strain has the ability of dissolving phosphorus. The strain RBC25 was screened and identified to have strong phosphorus dissolving ability (FIGS. 1A-1C).

Identification of dissolvability of aluminum and phosphorus: 5 ml of RBC25 bacterial solution with OD₆₀₀=1.0 was inoculated into 100 ml of mengjinna aluminum phosphorus liquid medium (Table 3), cultureD at 28° C., culture medium was sampled at different times, supernatant was obtained by centrifugation at 12000 rpm, finally the content of soluble phosphorus in the supernatant of liquid culture medium was determined by molybdenum antimony anti chromogenic method. The specific results are shown in FIG. 1C.

TABLE 2
Formulation of two kinds of bacterial phosphate solubilization solid
medium

| Medium | Compound | Amount/L | |
|---|---|---|---|
| Mengjinna | Glucose | 10 | g |
| organophosphorus bacteria | (NH₄)₂SO₄ | 0.5 | g |
| culture medium | NaCl | 0.2 | g |
| | MgSO_{4 •} 7H₂O | 0.3 | g |
| | FeSO_{4 •} 7H₂O | 0.01 | g |
| | MnSO₄ | 0.03 | g |
| | Yeast extract | 0.5 | g |
| | C₆H₆Ca₆O₂₄P₆ | 5.0 | g |
| | Agar | 20 | g |
| | pH: 7.0 | | |
| Mengjinna inorganic | Glucose | 10 | g |
| phosphorus bacteria | (NH₄)₂SO₄ | 0.5 | g |
| culture medium | NaCl | 0.2 | g |
| | MgSO_{4 •} 7H₂O | 0.3 | g |
| | FeSO_{4 •} 7H₂O | 0.01 | g |
| | MnSO₄ | 0.03 | g |
| | Yeast extract | 0.5 | g |
| | Ca₃(PO₄)₂ | 5.0 | g |
| | Agar | 20 | g |
| | pH: 7.0 | | |

TABLE 3
Formulation of Mengjinna liquid medium

| Medium | Compound | Amount/L | |
|---|---|---|---|
| Mengjinna Al—P medium | (NH₄)₂SO₄ | 0.5 | g |
| | Glucose | 10 | g |
| | NaCl | 0.3 | g |
| | KCl | 0.3 | g |
| | MgSO_{4 •} 7H₂O | 0.3 | g |
| | AlPO₄ | 15 | g |
| | FeSO_{4 •} 7H₂O | 0.03 | g |
| | MnSO_{4 •} 4H₂O | 0.03 | g |
| | Yeast extract | 0.4 | g |
| | pH: 7.0 | | |

### (3) Cultivation, Morphological and Biochemical Features of Strain RBC25

The phosphorus-dissolving bacterium RBC25 can grow on TSB medium and ordinary LB medium. On solid medium, its colony morphology is light yellow, the colony surface is glossy, the colony is round, and the colony boundary is clear. When strain RBC25 was cultured on solid medium of insoluble inorganic phosphorus and insoluble organic phosphorus, a transparent phosphorus dissolving ring will be formed around the colony.

### (4) 16S rDNA Sequence Analysis of Strain RBC25

The 16 rDNA of strain RBC25 was amplified by primers 16s-RNA-F (AGAGTTTGATCCTGGCTCAG, as shown in SEQ ID NO: 2) and 116s-RNA-R (TACGGCTACCTTGTTACGACTT, as shown in SEQ ID NO: 3) and sequenced (the specific sequence is shown in SEQ ID No: 1). The resulting sequences were submitted to NCBI (https://www.NCBINLM.NIH.Gov/) for blast alignment analysis and construction of evolutionary trees (FIG. 2). It is shown that strain RBC25 belongs to Burkholderia sp.

### EXAMPLE 2

### Colonization Analysis of Strain RBC25

(1) Vector pMG103-nptII-Luc containing firefly luciferase gene and vector pMG103-nptII-GFP were respectively transformed into strain RBC25 through electroporation method (12.5 kv/cm), positive clones were screened on Kan resistant LB plates, the phosphorolytic bacteria rbc25 labeled with firefly luciferase and green fluorescent protein were obtained.

(2) 10 ml OD₆₀₀=0.2 of the suspension containing firefly luciferase labeled strain RBC25 was inoculated into peat matrix and co cultured with soybean seedlings for 5 days for one week. The soybean plants were taken out from the substrate, the loose substrate on the root was shaken off, and the substrate of firefly luciferase was sprayed, the reaction time was 5 min, fluorescence signal acquisition was carried out in the imaging room of Tianneng chemiluminescence imaging instrument. Results as shown in FIG. 3, obvious chemical fluorescence signals can be detected in the root system of soybean, indicating that strain RBC25 can colonize all parts of soybean root system.

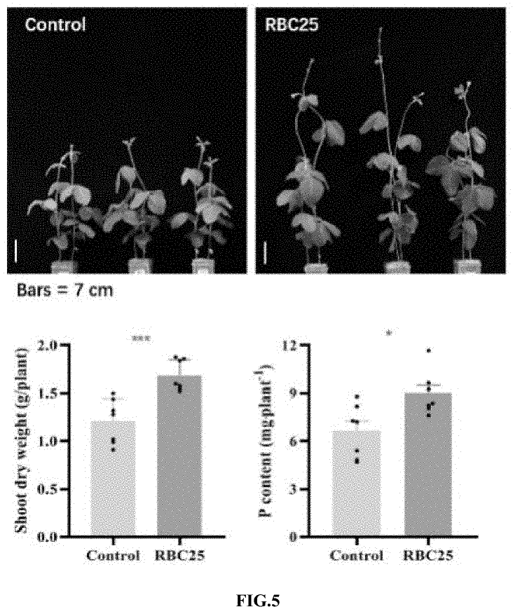

(3) 5 ml of strain RBC25 suspension carrying green fluorescent protein expression vector pMG103-nptII-GFP (OD₆₀₀=0.2) was sprayed onto 5 μM PI (see Table 4) and high phosphorus: 500 μM PI (see Table 5) on the hairy roots of MS medium, and co cultured at 28° C. for 5 days. Culture dishes with hairy roots were placed under a body fluorescence microscope and GFP signals were observed in a green fluorescence channel. The results (FIG. 4) showed that an obvious GFP fluorescence signal was detected on the roots of soybean hairy roots under low phosphorus conditions, while the GFP fluorescence signal was weak under high phosphorus conditions. The results showed that phosphorus deficiency signal (low phosphorus treatment) regulated the colonization of phosphorus bacteria rbc25 on soybean roots.

### EXAMPLE 3

### Effect of Inoculating Strain RBC25 Under Indoor Potted Condition for Biomass and P Nutrient Absorption in Soybean

(1) Strain activation: Strain RBC25 stored at −80° C. was inoculated into TSB solid medium and placed in a 28° C. incubator for culture. The activated strain was transferred to 5 ml of liquid TSB medium for culture after the growth of monoclonal antibody, placed in a shaker at 28° C. and 200 rpm, and cultured until the bacterial solution OD₆₀₀=1.0.

Bacterial solution 500 μL, was transferred to 50 ml liquid TSB medium and cultured at 28° C. for 2-3 days at 200 rpm until OD₆₀₀=1.5.

(2) The cells were collected by centrifugation (5000 rpm, 10 min), suspended in 50 ml sterile water, and adjusted to OD₆₀₀=0.2 with sterile water.

(3) Soybean seeds were planted in 7× seven× In 10 cm pots filled with sterilized (121° C., 30 min) growth substrate (Jiffy base peat (peat substrate: Netherlands JieFei company)) (the substrate is 1 cm away from the mouth of the basin), and 0.5 g of inorganic insoluble phosphorus Ca₃ (PO₄) 2 and 0.5 g of insoluble organic phosphorus calcium phytate (C₆H₆C₆O₂₄P₆) powder are added to the substrate of each basin.

(4) Four soybean seeds (Williams 82) were planted in each pot. Before planting, the soybean seeds were treated with surface treatment (70vol % alcohol for 1 minute). After the soybean leaves were fully expanded, the seedlings with uneven growth were removed, and only one plant was left in each pot. The experiment was set up with inoculation treatment and non inoculation treatment. The experimental group was inoculated with 10 ml of strain RBC25 with OD₆₀₀=0.2 per pot of substrate, and the control group was not inoculated with strain RBC25. The experimental group and the control group were planted with 6 pots of each treatment. During soybean growth, 50 ml of low phosphorus nutrient solution (5 μM PI) (Table 4). The soybean was co cultured with strain RBC25 in the growth chamber for 1 month, and then the growth of the soybean was photographed to determine the plant biomass and phosphorus content. The results showed (FIG. 5): under the condition of low phosphorus, compared with the control, inoculating strain RBC25 regulated by the phosphorus deficiency signal can significantly promote the growth of soybean, significantly increase the biomass and phosphorus absorption of soybean, in which the biomass of plant increased by 39.6%, and the total phosphorus absorption of plant increased by 35.4%. The results showed that inoculation of strain RBC25, a phosphate solubilizer regulated by phosphorus deficiency signal, promoted plant growth by increasing phosphorus uptake of soybean.

TABLE 4
Formulation of low phosphorus nutrient solution (5 μM Pi)

| | | | Concen- | | |
|---|---|---|---|---|---|
| Mother | | Sample | tration | 1 L | |
| liquor | Compound | weight g/L | times | (5 μM Pi) | |
| I | KNO₃ | 151.65 | 1000X | 1 | mL |
| | Ca(NO₃)₂ | 330.61 | | | |
| II | MgSO_{4 •} 7H₂O | 123.24 | 1000X | 1 | mL |
| | MnSO_{4 •} H₂O | 0.254 | | | |
| | ZnSO_{4 •} 7H₂O | 0.431 | | | |
| | CuSO_{4 •} 5H₂O | 0.125 | | | |
| | (NH₄)₆MO₇O_{24 •} 4H₂O | 0.2 | | | |
| | MgCl_{2 •} 6H₂O | 5.08 | | | |
| | CoCl₂ | 0.024 | | | |
| III | (NH₄)₂SO₄ | 66.148 | 1000X | 1 | mL |
| IV | EDTA (Na) _{•} Fe | 14.68 | 2000X | 0.5 | mL |
| V | Na₂B₄O₇ | 0.95 | 2000X | 0.5 | mL |
| VI | KH₂PO₄ | 68.05 | 2000X | 0.005 | mL |
| VII | K₂SO₄ | 52.28 | 1000X | 1 | mL |
| Low P | K₂SO₄ | 43.57 | 2000X | 0.495 | mL |
| supple- | | | | | |
| ment K | | | | | |

TABLE 5
Formulation of high phosphorus nutrient solution (500 μM Pi)

| | | Sample | Con- | | |
|---|---|---|---|---|---|
| Mother | | weight | centration | 1 L | |
| liquor | Compound | g/L | times | (5 μM Pi) | |
| I | KNO₃ | 151.65 | 1000X | 1 | mL |
| | Ca(NO₃)₂ | 330.61 | | | |
| II | MgSO_{4 •} 7H₂O | 123.24 | 1000X | 1 | mL |
| | MnSO_{4 •} H₂O | 0.254 | | | |
| | ZnSO_{4 •} 7H₂O | 0.431 | | | |
| | CuSO_{4 •} 5H₂O | 0.125 | | | |
| | (NH₄)₆MO₇O_{24 •} 4H₂O | 0.2 | | | |
| | MgCl_{2 •} 6H₂O | 5.08 | | | |
| | CoCl₂ | 0.024 | | | |
| III | (NH₄)₂SO₄ | 66.148 | 1000X | 1 | mL |
| IV | EDTA (Na) _{•} Fe | 14.68 | 2000X | 0.5 | mL |
| V | Na₂B₄O₇ | 0.95 | 2000X | 0.5 | mL |
| VI | KH₂PO₄ | 68.05 | 2000X | 0.005 | mL |
| VII | K₂SO₄ | 52.28 | 1000X | 1 | mL |

### EXAMPLE 4

### Effect of Inoculating Strain RBC25 Under Field Condition for Biomass and P Nutrient Absorption in Soybean

(1) Strain activation: Strain RBC25 stored at −80° C. was inoculated into TSB solid medium and placed in a 28° C. incubator for culture. The activated strain was transferred to 5 ml of liquid TSB medium for culture, placed in a shaker at 28° C. and 200 rpm, and cultured until the bacterial solution OD₆₀₀=1.5.

(2) Bacterial solution 500 μL was transferred to 50 ml liquid TSB medium and cultured at 28° C. for 2-3 days at 200 rpm until OD₆₀₀=1.5.

(3) The cells were collected by centrifugation (5000 rpm, 10 min), suspended in 50 ml sterile water, and adjusted to OD₆₀₀=1.0 with sterile water.

(4) 50 ml of bacteria solution with OD₆₀₀=1.0 after resuspending and 20 g of plant growth medium (Jiffy base peat/Netherland geffe company) was mixed in a volume ratio of 1:1 to prepare the bacteria agent. The experiment was set up with no inoculant as the control.

(5) Field experiment planning: Experimental settings of each cell: 5 meters long and 1.2 meters wide. The plant spacing of soybean was 20 cm and the row spacing was 40 cm. Sow 2 seeds per hole. The experiment group was inoculated with the phosphate dissolving bacteria rbc25. Before sowing, the soybean seeds were mixed with the prepared bacteria. The experiment was carried out without inoculating bacteria. The experiment was divided into the experimental group and the control group, and three plots were set up respectively.

During cultivation, soybean shall be watered normally according to the demand, and no fertilizer shall be applied. Soybeans were harvested at pod filling stage, and total phosphorus absorption and biomass of soybeans were tested.

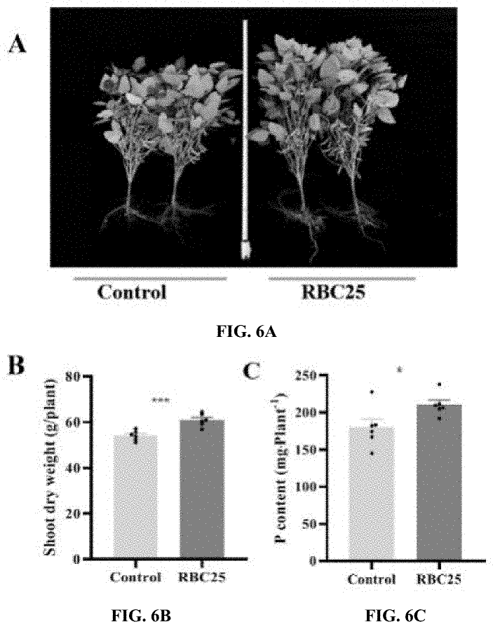

(6) The result is shown in FIGS. 6A-6C: Inoculating phosphate-solubilizing bacteria RBC25 regulated by Phosphate deficiency in the field can promote the growth of soybean. Inoculation with strain RBC25 increased biomass by 12.5% compared to the non-inoculated control. Inoculation with strain RBC25 increased total phosphorus uptake by 16.9% compared to the non-inoculated control. That indicates inoculating phosphate-solubilizing bacteria RBC25 regulated by Phosphate deficiency can promote the growth and phosphorus absorption of soybean. It suggested of a favourable prospect of application.

The above examples are only preferred embodiments of the present invention. All equal changes and modifications made according to the scope of the patent application of the present invention shall belong to the scope of the present invention.

What is claimed is:

1. A phosphorus-dissolving bacterium RBC25 regulated by a Phosphate deficiency, wherein the phosphorus-dissolving bacterium RBC25 is isolated from roots of a soybean planted in an acidic soil and is taxonomically classified as Burkholderia sp. RBC25; wherein the phosphorus-dissolving bacterium RBC25 is deposited on Jun. 28, 2021 in the China Center for Type Culture Collection in Wuhan University, Wuhan City, Hubei Province with a deposit number of CCTCC NO: M2021796.

2. The phosphorus-dissolving bacterium RBC25 regulated by the Phosphate deficiency according to claim 1, wherein the phosphorus-dissolving bacterium RBC25 is regulated by the phosphate deficiency and is configured to be enriched in the roots of the soybean under phosphate-deficient conditions.

3. A method of an application of the phosphorus-dissolving bacterium RBC25 regulated by the Phosphate deficiency according to claim 1 in promoting a soybean growth.
